(19) United States
(12) Patent Application Publication
(10) Pub. No.: US 2016/0339358 A1
(43) Pub. Date: Nov. 24, 2016

(54) MANUFACTURING AND SEPARATING DEVICE FOR OILY RADIOACTIVE SUBSTANCES AND METHOD THEREOF

(71) Applicant: Institute of Nuclear Energy Research, Atomic Energy Council, Executive Yuan, R.O.C., Taoyuan (TW)

(72) Inventors: CHIH-CHING WANG, Taoyuan (TW); TSAI-YUEH LUO, Taoyuan County (TW); I-CHUNG TANG, Taoyuan (TW)

(21) Appl. No.: 15/148,407

(22) Filed: May 6, 2016

(30) Foreign Application Priority Data
May 22, 2015 (TW) 104116540

(51) Int. Cl. B01D 17/02; B01J 14/00; B01D 17/12; A61K 51/04

(52) U.S. Cl. CPC B01D 17/0202; A61K 51/0404; B01J 14/00; B01D 17/12

(57) ABSTRACT

A manufacturing and separating device and a method for oily radioactive substances provide an automatic synthesis box to minimize the man-made errors during the manufacturing, such that mass production for the oily radioactive substance can be possible, the radiation exposure time of the manufacturing staff can be reduced, and the radioactivity of the final products can be detected. The manufacturing and separating device includes a first reaction material bottle, a first heating device, a gas pump, a first flushing material bottle, a second flushing material bottle, a purifying device, a first transmission unit, a temperature-controlled temporary storage tank, a filter, a first collection bottle, a second collection bottle, a plurality of valves and a control unit.

### CROSS REFERENCE TO RELATED APPLICATION

This application claims the benefit of Taiwan Patent Application Serial No. 104116540, filed May 22, 2015, the subject matter of which is incorporated herein by reference.

### BACKGROUND OF INVENTION

1. Field of the Invention

The invention relates to a device and a method for manufacturing and separating oily radioactive substances, and more particularly to a device and a manufacturing method for a hepatic artery injection agent.

2. Description of the Prior Art

According to statistics of the health promotion administration of the ministry of health and welfare for the year of 2012, the cancers rank first among the ten major causes of death in Taiwan, and particularly the lung cancer ranks second among the cancers. Thus, the institute of nuclear energy research adopts merits of the clinical hepatic artery injection agent “lipiodol” and the brachy therapy of radioisotopes to develop the new hepatic artery injection agent “188Re-MN-16ET/Lipiodol” for attaining objects of dual-effect and tracking, and the 188Re-MN-16ET/Lipiodol has been patented in Taiwan in 2012 (Taiwan Patent No. 1355277). Currently, most of the manufacturing processes for the hepatic artery injection agents are all manual operated, and thus can't be arranged for mass production.

Further, since, in the manufacturing processes of the hepatic artery injection agent, pharmaceutical personals or manufacturing staff would increase the radiation exposure with the increase in radioactivity and currently the manufacturing processes for the 188Re-MN-16ET/Lipiodol are mainly manual operated, thus plenty man-made errors would involve during the manufacturing. While in meeting problems in yield or products, the major reason is hard to be located, and definitely the commercialization thereof and the following mass production would be retarded. It is obvious that the bottle-neck issue is the manual operation in the conventional manufacturing or preparing processes. In view of radioactive protection, during a the conventional preparing process for the hepatic artery injection agents, the manufacturing staff needs to be exposed under the radiation for a long period, and the exposure time would increase significantly with the production quantity and the radioactivity. To resolve the obstacle for mass production in manufacturing the hepatic artery injection agents and the jeopardy in radiation exposure for the manufacturing staff, an effort by introducing an automatic synthesis box is provided in this invention.

### SUMMARY OF THE INVENTION

Accordingly, it is the primary object of the present invention to provide a manufacturing and separating device and a method for oily radioactive substances that provides an automatic synthesis box to minimize the man-made errors during the manufacturing, such that mass production for the oily radioactive substance can be possible. In addition, the automatic synthesis box can reduce the radiation exposure time of the manufacturing staff, and can detect the activity of the final products. Further, by introducing a filter-membrane pressure-detecting system to the device, possible leakage problems can thus be avoided. Furthermore, in the device of the present invention, an automatic cleaning mode is also provided.

In the present invention, the manufacturing and separating device for oily radioactive substances comprises:

a first reaction material bottle, containing a first raw material;

a first heating device, having thereinside a second reaction material bottle containing a second raw material and receiving the first raw material, heating and mixing the first raw material and the second raw material so as to form a reacted material;

a gas pump, sending a gas into the second reaction material bottle of the first heating device for stirring the first raw material and the second raw material;

a first flushing material bottle, containing an aqueous cleaning material;

a second flushing material bottle, containing an oily cleaning material;

a purifying device, including thereinside an oily absorbing structure for adhering the reacted material, outputting an oily substance and an aqueous substance;

a first transmission unit, leading out the aqueous cleaning material and the oily cleaning material to the purifying device, forwarding the reacted material in the first heating device to the purifying device;

a temperature-controlled temporary storage tank, receiving the oily substance and the aqueous substance outputted from the purifying device, having thereinside a second heating device for heating the oily substance and the aqueous substance;

a filter, receiving the oily substance and the aqueous substance from the temperature-controlled temporary storage tank, allowing the oily substance to pass through, blocking the aqueous substance;

a first collection bottle, collecting the oily substance from the filter;

a second collection bottle, collecting the oily substance from the purifying device;

a second transmission unit, conveying the oily substance from the purifying device to the second collection bottle;

a plurality of valves, opening or closing corresponding connection piping among the first reaction material bottle, the first flushing material bottle, the second flushing material bottle, the first transmission unit, the second transmission unit, the third transmission unit, the first heating device, the purifying device, the temperature-controlled temporary storage tank and the first collection bottle; and

a control unit, connecting the plurality of valves so as to control opening and closing of the valves.

In the present invention, the manufacturing and separating method for oily radioactive substances comprises:

a step of heating a first heating device to a predetermined temperature;

a step of sending a raw material to a second reaction material bottle of the first heating device;

a step of applying a gas pump to send a gas into the second reaction material bottle so as to stir the raw materials thereinside, and to send another gas to cool the heated first heating device;

a step of applying a first transmission unit to convey the reacted material inside the first heating device to the purifying device, to lead out the aqueous cleaning material inside first heating device to the waste-liquid recycling bottle via the purifying device, and also to lead out the oily cleaning material inside the first heating device to a temperature-controlled temporary storage tank via the purifying device;

a step of applying the temperature-controlled temporary storage tank to receive the oily substance and a few aqueous substance outputted from the purifying device, wherein the temporary storage tank has thereinside a second heating device for heating the oily substance and the aqueous substance;

a step of applying a filter to receive the oily substance and a few aqueous substance outputted from the temperature-controlled temporary storage tank, wherein the filter allows the oily substance to pass through, but blocks the aqueous substance, wherein the oily substance passing through the filter is then collected by a first collection bottle;

a step of applying a second transmission unit to convey the oily substance outputted from the purifying device to a second collection bottle; and

a step of applying a third transmission unit and a third flushing material bottle containing thereinside a volatile material to flush all the aforesaid members and the connection piping.

All these objects are achieved by the manufacturing and separating device for oily radioactive substances and a method thereof described below.

### BRIEF DESCRIPTION OF THE DRAWINGS

The present invention will now be specified with reference to its preferred embodiment illustrated in the drawings, in which:

FIG. 1 is a schematic view of a manufacturing and separating device for oily radioactive substances in accordance with the present invention; and

FIG. 2 is a flowchart of a manufacturing and separating method for oily radioactive substances in accordance with the present invention.

### DESCRIPTION OF THE PREFERRED EMBODIMENT

The invention disclosed herein is directed to a manufacturing and separating device for oily radioactive substances and a method thereof. In the following description, numerous details are set forth in order to provide a thorough understanding of the present invention. It will be appreciated by one skilled in the art that variations of these specific details are possible while still achieving the results of the present invention. In other instance, well-known components are not described in detail in order not to unnecessarily obscure the present invention.

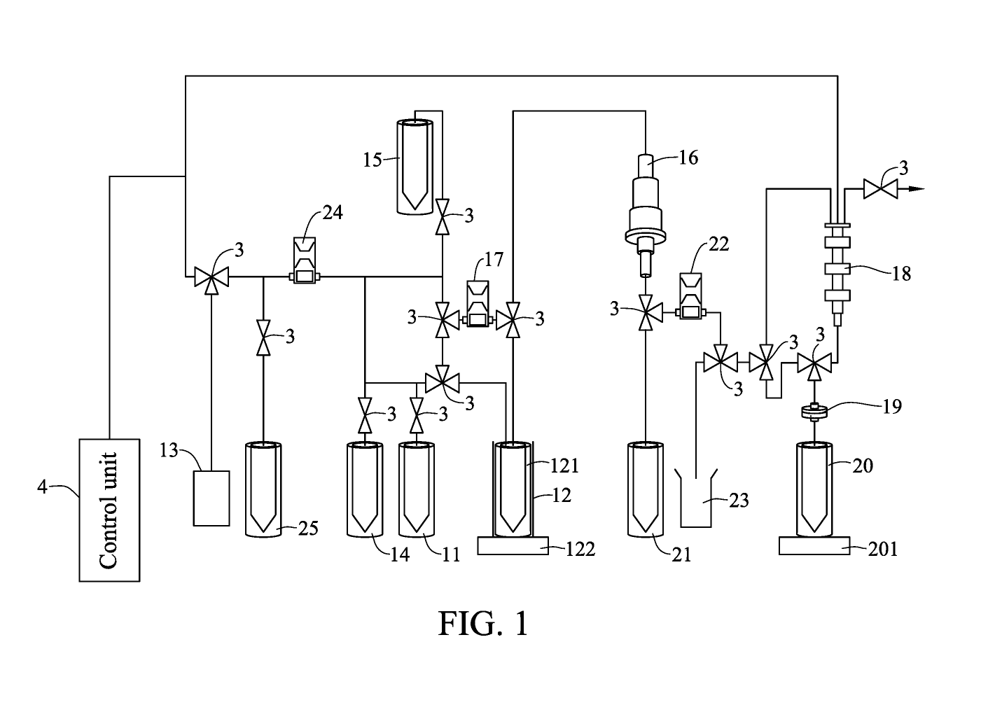

Referring now to FIG. 1, the manufacturing and separating device for oily radioactive substances in accordance with the present invention includes a first reaction material bottle 11, a first heating device 12, a gas pump 13, a first flushing material bottle 14, a second flushing material bottle 15, a purifying device 16, a first transmission unit 17, a temperature-controlled temporary storage tank 18, a filter 19, a first collection bottle 20, a second collection bottle 21, a plurality of valves 3, and a control unit 4. The first reaction material bottle 11 contains a raw material. The first heating device 12 further has thereinside a second reaction material bottle 121 containing another raw material and receiving the aforesaid raw material, and is to heat and mix the two raw materials so as to form a reacted material. A bottom of the first heating device 12 further has a first radioactivity sensor 122. The gas pump 13 is to send a gas into the second reaction material bottle 121 for stirring the raw materials, to send a gas into the first heating device 12 for a cooling purpose, and to forward the substances inside the temperature-controlled temporary storage tank 18 to the first collection bottle 20 by passing the filter 19. The first flushing material bottle 14 contains an aqueous cleaning material. The second flushing material bottle 15 contains an oily cleaning material. The purifying device 16 further includes thereinside an oily absorbing structure (not shown in the figure, formed by a plurality of leaf-shape plates) for adhering the reacted material, which the oily absorbing structure can absorb lipophilic substances. The first transmission unit 17 forwards the reacted material in the second reaction material bottle 121 of the first heating device 12 to the purifying device 16, and also lead out the aqueous cleaning material and the oily cleaning material and sends them to the waste-liquid recycling bottle 23 and the temperature-controlled temporary storage tank 18, respectively, via the purifying device 16. The temperature-controlled temporary storage tank 18 receives the oily substance and a few aqueous substance from the purifying device 16. Further, the temperature-controlled temporary storage tank 18 has an interior second heating device for heating the oily substance and the aqueous substance. The filter 19 receives the oily substance and the aqueous substance from the temperature-controlled temporary storage tank 18 but allows the oily substance only to pass through. Namely, the aqueous substance is blocked by the filter 19. The first collection bottle 20 for collecting the oily substance from the filter 19 further includes a second radioactivity sensor 201 located at a bottom thereof. The second collection bottle 21 is to collect the oily substance from the purifying device 16. The second transmission unit 22 is to convey the oily substance from the purifying device 16 to the second collection bottle 21. The valves 3 are to open or close the connection piping among the first reaction material bottle 11, the first flushing material bottle 14, the second flushing material bottle 15, the first transmission unit 17, the second transmission unit 22, the third transmission unit 24, the first heating device 12, the purifying device 16, the temperature-controlled temporary storage tank 18 and the first collection bottle 20. The control unit 4 connecting the valves 3 is to control the open/close of the valves 3.

The aforesaid raw material can be consisted of a perrhenate solution, an H3-MN-16ET, a stannous and a tartaric acid.

The aforesaid aqueous cleaning material contained in the first flushing material bottle 14 can be saline water. The oily cleaning material contained in the second flushing material bottle 15 can be a contrast medium.

In the present invention, the manufacturing and separating device for the oily radioactive substances can further include a third transmission unit 24 and a third flushing material bottle 25. The third flushing material bottle 25 contains a volatile material. The third transmission unit 24 and the third flushing material bottle 25 is used to flush the first reaction material bottle 11, the first flushing material bottle 14, the second flushing material bottle 15, the first transmission unit 17, the second transmission unit 22, the third transmission unit 24, the first heating device 12, the purifying device 16, the temperature-controlled temporary storage tank 18 and all the connection piping among the aforesaid members. The volatile material can be an alcohol.

The aforesaid second heating device can further connect an exhaust valve.

The aforesaid second transmission unit 22 and the temperature-controlled temporary storage tank 18 can further connect a waste-liquid recycling bottle 23 for collecting the additional aqueous substance outputted from the purifying device 16 and the temperature-controlled temporary storage tank 18.

The aforesaid control unit 4 for performing parameter setting and automatic control can be a personal computer, a notebook computer, a tablet computer or a smart phone.

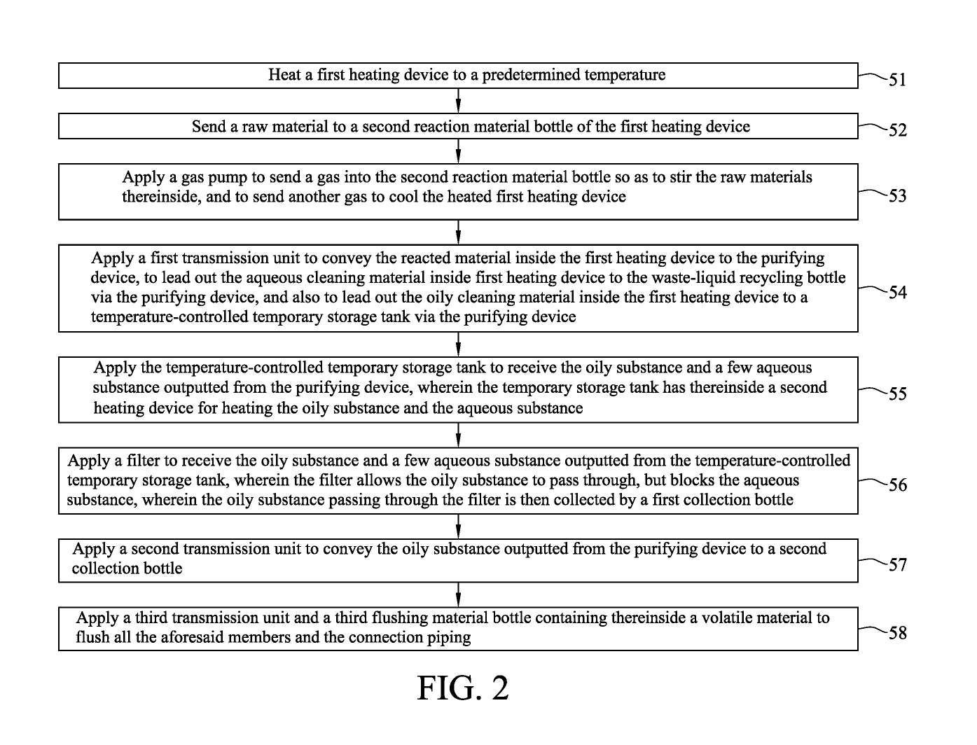

Referring now to FIG. 2 and also to FIG. 1, a flowchart of the manufacturing and separating method for oily radioactive substances in accordance with the present invention is shown. The method includes the following steps.

Step 51: Heat a first heating device 12 to a predetermined temperature.

Step 52: Send a raw material to a second reaction material bottle 121 of the first heating device 12.

Step 53: Apply a gas pump 13 to send a gas into the second reaction material bottle 121 so as to stir the raw material thereinside, and to send another gas to cool the heated first heating device 12.

Step 54: Apply a first transmission unit 17 to convey the reacted material inside the first heating device 12 to the purifying device 16, to lead out the aqueous cleaning material inside first heating device 12 to the waste-liquid recycling bottle 23 via the purifying device 16, and also to lead out the oily cleaning material inside the first heating device 12 to a temperature-controlled temporary storage tank 18 via the purifying device 16.

Step 55: Apply the temperature-controlled temporary storage tank 18 to receive the oily substance and a few aqueous substance outputted from the purifying device 16, in which the temporary storage tank 18 has thereinside a second heating device (not shown in the figure) for heating the oily substance and the aqueous substance.

Step 56: Apply a filter 19 to receive the oily substance and a few aqueous substance outputted from the temperature-controlled temporary storage tank 18, in which the filter 19 allows the oily substance to pass through, but blocks the aqueous substance. The oily substance passing through the filter 19 is then collected by a first collection bottle 20.

Step 57: Apply a second transmission unit 22 to convey the oily substance outputted from the purifying device 16 to a second collection bottle 21.

Step 58: Apply a third transmission unit 24 and a third flushing material bottle 25 containing thereinside a volatile material to flush all the aforesaid members and the connection piping.

As shown in FIG. 1 and FIG. 2, it is understood that the manufacturing and separating device and method for the oily radioactive substances in accordance with the present invention are mainly to provide an automatic synthesis box so as to minimize possible man-made errors during the manufacturing processes. Upon such an arrangement, mass production can be possible, and labors for operations can be reduced. Further, the automatic synthesis box can shorten the radiation exposure time to the manufacturing staff, and can detect the radioactivity of the final product. In the device, a filter-membrane pressure-detecting system is included to avoid leakage. Further, the device of the present invention can perform an automatic cleaning. For all the aforesaid advantages of the present invention over the prior art, it is believed that the device and the method provided herein shall be patentable.

While the present invention has been particularly shown and described with reference to a preferred embodiment, it will be understood by those skilled in the art that various changes in form and detail may be without departing from the spirit and scope of the present invention.

What is claimed is:

1. A manufacturing and separating device for oily radioactive substances, comprising:
a first reaction material bottle, containing a first raw material;
a first heating device, having thereinside a second reaction material bottle containing a second raw material and receiving the first raw material, heating and mixing the first raw material and the second raw material so as to form a reacted material;
a gas pump, sending a gas into the second reaction material bottle of the first heating device for stirring the first raw material and the second raw material;
a first flushing material bottle, containing an aqueous cleaning material;
a second flushing material bottle, containing an oily cleaning material;
a purifying device, including thereinside an oily absorbing structure for adhering the reacted material, outputting an oily substance and an aqueous sub stance;
a first transmission unit, leading out the aqueous cleaning material and the oily cleaning material to the purifying device, forwarding the reacted material in the first heating device to the purifying device;
a temperature-controlled temporary storage tank, receiving the oily substance and the aqueous substance outputted from the purifying device, having thereinside a second heating device for heating the oily substance and the aqueous sub stance;
a filter, receiving the oily substance and the aqueous substance from the temperature-controlled temporary storage tank, allowing the oily substance to pass through, blocking the aqueous substance;
a first collection bottle, collecting the oily substance from the filter;
a second collection bottle, collecting the oily substance from the purifying device;
a second transmission unit, conveying the oily substance from the purifying device to the second collection bottle;
a plurality of valves, opening or closing corresponding connection piping among the first reaction material bottle, the first flushing material bottle, the second flushing material bottle, the first transmission unit, the second transmission unit, the third transmission unit, the first heating device, the purifying device, the temperature-controlled temporary storage tank and the first collection bottle; and
a control unit, connecting the plurality of valves so as to control opening and closing of the valves.

2. The manufacturing and separating device for oily radioactive substances of claim 1, wherein the first raw material is consisted of a perrhenate solution, and the second reaction material is consisted of an H3-MN-16ET, a stannous and a tartaric acid.

3. The manufacturing and separating device for oily radioactive substances of claim 1, wherein the aqueous cleaning material contained in the first flushing material bottle is saline water.

4. The manufacturing and separating device for oily radioactive substances of claim 1, wherein the oily cleaning material contained in the second flushing material bottle is a contrast medium.

5. The manufacturing and separating device for oily radioactive substances of claim 1, further including a third transmission unit and a third flushing material bottle, the third flushing material bottle containing a volatile material, the third transmission unit and the third flushing material bottle being to flush the first reaction material bottle, the first flushing material bottle, the second flushing material bottle, the first transmission unit, the second transmission unit, the third transmission unit, the first heating device, the purifying device, the temperature-controlled temporary storage tank and the connection piping.

6. The manufacturing and separating device for oily radioactive substances of claim 5, wherein the volatile material is an alcohol.

7. The manufacturing and separating device for oily radioactive substances of claim 1, wherein the second heating device further connects an exhaust valve.

8. The manufacturing and separating device for oily radioactive substances of claim 1, wherein the second transmission unit connects a waste-liquid recycling bottle for collecting the additional aqueous substance outputted from the purifying device.

9. The manufacturing and separating device for oily radioactive substances of claim 1, wherein the control unit is one of a personal computer, a notebook computer, a tablet computer and a smart phone.

10. The manufacturing and separating device for oily radioactive substances of claim 1, wherein the oily absorbing structure is formed by a plurality of leaf-shape plates.

11. The manufacturing and separating device for oily radioactive substances of claim 1, wherein the first heating device further includes a first radioactivity sensor located at a bottom thereof.

12. The manufacturing and separating device for oily radioactive substances of claim 1, wherein the first collection bottle further includes a second radioactivity sensor located at a bottom thereof.

13. A manufacturing and separating method for oily radioactive substances, comprising the steps of:
(A) heating a first heating device to a predetermined temperature;
(B) sending a raw material to a second reaction material bottle of the first heating device;
(C) applying a gas pump to send a gas into the second reaction material bottle so as to stir the raw materials thereinside, and to send another gas to cool the heated first heating device;
(D) applying a first transmission unit to convey the reacted material inside the first heating device to the purifying device, to lead out the aqueous cleaning material inside first heating device to the waste-liquid recycling bottle via the purifying device, and also to lead out the oily cleaning material inside the first heating device to a temperature-controlled temporary storage tank via the purifying device;
(E) applying the temperature-controlled temporary storage tank to receive the oily substance and a few aqueous substance outputted from the purifying device, wherein the temporary storage tank has thereinside a second heating device for heating the oily substance and the aqueous substance;
(F) applying a filter to receive the oily substance and a few aqueous substance outputted from the temperature-controlled temporary storage tank, wherein the filter allows the oily substance to pass through, but blocks the aqueous substance, wherein the oily substance passing through the filter is then collected by a first collection bottle; and
(G) applying a second transmission unit to convey the oily substance outputted from the purifying device to a second collection bottle.

14. The manufacturing and separating method for oily radioactive substances of claim 13, further including a step (H) of applying a third transmission unit and a third flushing material bottle containing thereinside a volatile material to flush all the aforesaid members and the connection piping.

15. The manufacturing and separating method for oily radioactive substances of claim 13, wherein a control unit is applied to perform parameter setting and automatic control, wherein the control unit is one of a personal computer, a notebook computer, a tablet computer and a smart phone.

16. The manufacturing and separating method for oily radioactive substances of claim 13, wherein the volatile material is an alcohol.

17. The manufacturing and separating method for oily radioactive substances of claim 13, wherein the raw material is consisted of a perrhenate solution, an H3-MN-16ET, a stannous and a tartaric acid.

18. The manufacturing and separating method for oily radioactive substances of claim 13, wherein the aqueous cleaning material contained in the first flushing material bottle is saline water.

19. The manufacturing and separating method for oily radioactive substances of claim 13, wherein the oily cleaning material contained in the second flushing material bottle is a contrast medium.

20. The manufacturing and separating method for oily radioactive substances of claim 13, wherein the second heating device further connects an exhaust valve.

21. The manufacturing and separating method for oily radioactive substances of claim 13, wherein the second transmission unit and the temperature-controlled temporary storage tank further connect a waste-liquid recycling bottle for collecting the additional aqueous substance outputted from the purifying device and the temperature-controlled temporary storage tank.
